(19) United States
(12) Patent Application Publication
(10) Pub. No.: US 2024/0409623 A1
(43) Pub. Date: Dec. 12, 2024

(54) Polyclonal Ovine Fab Raised Against Ricin

(71) Applicant: BTG International Inc., Conshohocken, PA (US)

(72) Inventors: Dafydd Aled GRIFFITHS, Pothyrhyd (GB); Christon HILL, Wayland, MA (US); Ellen Dilys DENTTEN, Newcastle Emlyn (GB); Zainab Angela BASCAL, Waterlooville (GB)

(21) Appl. No.: 18/741,323

(22) Filed: Jun. 12, 2024

Related U.S. Application Data
(60) Provisional application No. 63/472,469, filed on Jun. 12, 2023.

(51) Int. Cl. C07K 16/16; A61K 39/00; A61P 39/02

(52) U.S. Cl. CPC C07K 16/16; A61K 39/00; A61P 39/02

(57) ABSTRACT

The disclosure relates to a pharmaceutical composition, having as an active ingredient isolated antigen-binding fragments obtained from ovine and capable of inhibiting/neutralizing ricin, or variant thereof, as well as, methods of making and use thereof.

### CROSS-REFERENCE TO RELATED APPLICATIONS

This application claims the benefit of U.S. Provisional Patent Application Ser. No. 63/472,469, filed Jun. 12, 2023, the entire contents of which are incorporated by reference herein.

### TECHNICAL FIELD

The present disclosure relates to molecules which bind to and treat ricin poisoning, and more particularly, to polyclonal antibody fragments.

### BACKGROUND

Ricin is a plant-derived toxin isolated from seeds of the castor oil plant Ricinus communis and is rated by the United States Centers for Disease Control and Prevention as a category B bioterrorism agent. Death from ricin poisoning can occur within 36-72 hours of exposure and there is currently no approved therapy for ricin intoxication.

### SUMMARY

Embodiments of the disclosure include a pharmaceutical composition, having as an active ingredient isolated antigen-binding fragments obtained from ovine serum and capable of inhibiting/neutralizing ricin.

Additional embodiments of the disclosure include a method of treating ricin poisoning, by administering a pharmaceutically effective amount of the composition of a pharmaceutical composition, having as an active ingredient isolated antigen-binding fragments obtained from ovine serum and capable of inhibiting/neutralizing ricin.

Other embodiments of the disclosure include a method, involving immunizing a sheep with ricin or ricin antigen; collecting blood from the sheep after the immunizing; preparing a serum from the blood obtained in the collecting; and purifying the serum to obtain isolated antigen-binding fragments.

### BRIEF DESCRIPTION OF DRAWINGS

FIG. 1 is a flow chart showing a manufacturing process.

FIG. 2 shows the survival rate of mice exposed ricin and FABs disclosed herein.

FIG. 3 shows body weight changes of mice exposed ricin and FABs disclosed herein.

### DETAILED DESCRIPTION

Before certain embodiments are described in greater detail, it is to be understood that this disclosure is not limited to certain embodiments described, as such may, of course, vary. It is also to be understood that the terminology used herein is for the purpose of describing certain embodiments only, and is not intended to be limiting, since the scope of the present disclosure will be limited only by the appended claims.

Described herein are several definitions. Such definitions are meant to encompass grammatical equivalents.

The use of “or” means “and/or” unless stated otherwise. Furthermore, the use of the terms “comprising,” “having,” “including,” as well as other forms, such as “includes” and “included,” are intended to be inclusive and mean that there may be additional elements other than the listed elements.

As used herein, the term “about” when referring to a measurable value such as an amount, a temporal duration, and the like, is meant to encompass variations. Such variations, however, are dependent on the specific component referred to and the context as understood by a person of ordinary skill in the art.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, representative illustrative methods, and materials are now described.

Each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible.

In general, the polyclonal antibodies of the present disclosure are prepared when animals are immunized to produce a range of antibodies specific to a specific antigen. These proteins are extracted from the serum and purified, then cleaved to separate the fragment that binds to the antigen (fab or FAB) from the Fc, or fragment crystallizable region. These smaller fragments can access difficult-to-reach therapeutic targets and may be less likely to cause unwanted downstream effects on the immune system. Additional filtration and viral clearance stages help to ensure purity and avoid adverse reactions.

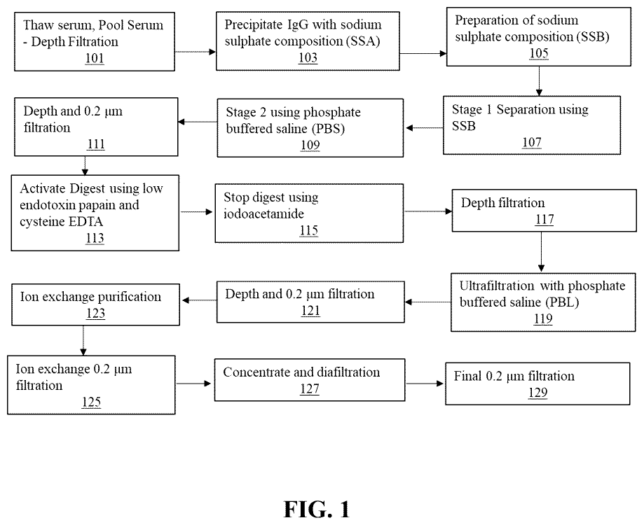

To obtain anti-ricin Fab fragments, serum generated from sheep immunized with a Ricinus communis-derived protein was purified using a polyclonal ovine antibody platform shown in FIG. 1.

In general, the process for preparing polyclonal antibodies involves a step of animal selection. Animals relevant to polyclonal antibody production include chickens, goats, guinea pigs, hamsters, horses, mice, rats, camels, llamas/alpaca, and ovine. Consistent with the polyclonal antibodies disclosed herein, the animal is preferably ovine. Animal selection is typically based on the amount of antibody required; the relationship between antigen and animal; and necessary characteristics of the antibody to be made. For instance, goats or horses may be used if larger quantities of antisera are necessary. Alternatively, many programs utilize chickens because egg yolks contain IgY (IgG) and eliminate the need for bleeding. However, chicken IgY does not perform as a precipitating antibody using standard solutions. The selection of animal may impact the final polyclonal antibody.

Examples of ovine breeds include Droper, Valais Blacknose, Merino, Suffolk, Awassi, Cameroon, Texel, Priangan, Ouessant, Sardi, Romanov, Harri, Arabi, Scottish Blackface, Hampshire Down, Katahdin, Dorset, Southdown, Corriedale, Herdwick, Najdi, Ladoum, Racka, Soay, Polypay, Fries Melkschaap, Lacune, Pelibuey, Montadale, Marco Polo, Beltex, Zwarbles, Barbados Black, Ryeland, Jacob, Navajo-Churro, American Blackbelly, Shropshire, Shetland, Meatmaster, Ile-de-France, Wiltshire Horn, badger Face Welsh Mountain, Wiltipoll, Rambouillet, Poll Dorset, Lonk, Cheviot, Columbia, Australian White, and Romney.

In addition to animal selection, antigen preparation can affect both the quality and the quantity of antibody produced. The selection of antigen is such that it does not induce tolerance, suppression, or immune deviation instead of a humoral response. In general, antigens of the present disclosure are Ricinus communis-derived proteins, preferably from seeds of Castor bean plant (Ricinus communis). The Ricinus communis-derived proteins can be natural or synthetic.

Examples of various Ricinus communis-derived proteins include the various isoforms of ricin. In general, the isoforms of ricin have differential glycosylation depending on the seed variety, however, other differences may exist depending on specific isoforms being compared. Examples of different forms of Ricinus communis-derived proteins include ricin isoforms D and E, and the agglutinin RCA120. Other isoforms include ricin toxin A (RTA) having a molecular weight of 32 kDa and ricin toxin B (RTB) having a molecular weight of 34 kDA. RTA and RTB refer to the two functionally different polypeptide chains on the ricin toxin. In certain embodiments, the Ricinus communis-derived proteins are conjugated or crosslinked to larger, immunogenic, carrier proteins. Accordingly, Ricinus communis-derived proteins can have a weight greater than, for example, about 32 kDa; about 34 kDa, about 65 kDa; or about 120 kDa.

To immunize ovine for antibody production, the sheep can be administered the Ricinus communis-derived protein via injection. In general, the Ricinus communis-derived proteins will include an adjuvant or carrier. The amount of Ricinus communis-derived proteins ranges from micrograms to milligrams and will vary based on the selected Ricinus communis-derived protein. The adjuvant is selected based on compatibility with the specific animal and antigen. Examples of adjuvants include Freund's adjuvant; Ribi Adjuvant Systems (RAS); Hunter's TiterMax®; Montanide ISA Adjuvants®; and Syntex Adjuvant Formulation (SAF)®.

In certain embodiments, the antigen is purified prior to mixing with the adjuvant. Purification can include filtration (for example a 0.22 μm filter) and the removal of polyacrylamide gel, SDS, urea, endotoxin, and particulate matter. The antigen is preferably maintained at a normal pH. In general, the preparation for injection is free of other solvents or potentially toxic agents. The antigen can optionally include sterile saline.

In specific embodiments, the antigen and adjuvant are prepared as an emulsion for injection.

Immunization can be performed via a single administration or multiple administrations. Multiple administration can include, for example, about 2-15 injections, 3-10 injections, or about 4-8 injections. In embodiments having multiple administrations via injection, injections can be spaced at least 1-3 inches apart. Additional steps for administration can include sterilization and inspection of the site of administration. Injection sites can be prepared via shaving and sterilization with betadine and alcohol. Injections are administered subcutaneously. Following immunization, ovine can be monitored. Monitoring can be daily, and includes review of: pain, swelling, abscessation, fistula formation, infection, and/or ulceration at or near the immunization site(s).

Immunization can occur over multiple doses. A first immunization dose is a priming dose and may be followed by booster doses. Booster doses are approximately about 50% the priming dose. In certain embodiments, the immunization includes a secondary immunization that is about 28 days or about one-month after the first immunization.

Following immunization, blood is collected from the sheep. Blood collection can be less than 1.25% (1.25 ml/100 g) of the animal's current body weight over a two-week period. The blood collection may occur in a single draw or over multiple draws. The animal may be monitored for anemia. Anemia may be monitored via assays such as hematocrit and/or serum protein levels. In some embodiments, animals are under anesthesia prior to blood collection.

Serum is prepared from the whole blood collected from the animal. Serum can be prepared by allowing the collected blood to clot. Clotting can be achieved by leaving the collected blood undisturbed at room temperature. The clots can be separated from the blood via centrifugation. In some embodiments, centrifugation is at 1,000-2,000×g for 10 minutes. The centrifuge is optionally refrigerated.

The supernatant, or serum, following centrifugation can be transferred to a new container and stored for future use. The serum can be maintained at a temperature between 2-8° C. during handling and −20° C. or lower for storage and transportation.

Isolated antigen-binding fragments are obtained by purifying the serum. Purification of the serum is illustrated in FIG. 1.

Purification can include a first step 101 of thawing stored serum. Thawed serum is then pooled together to form a polyclonal containing composition. Following pooling, the polyclonal containing composition is run through a depth filter to capture any solid contaminants.

In a second step 103, a first composition of sodium sulphate (SSA) is added to the polyclonal containing composition to precipitate the IgG. Other sulfates can be used to precipitate the IgG, for example ammonium sulfate.

In a third step 105, a second composition of sodium sulphate (SSB) is prepared. The second composition of sodium sulphate (SSB) can be different from the first composition of sodium sulphate (SSA). In a fourth step 107, the second composition of sodium sulphate (SSB) is used in a first stage of separation (Stage 1) of the polyclonal containing composition. Following the Stage 1 separation, a fifth step 109 of separating the polyclonal antibodies in the polyclonal containing composition can be preformed using phosphate buffered saline (PBS) (Stage 2).

Once separated, a sixth step 111 of filtration can be performed on the polyclonal containing composition. The filtration of the sixth step is preferably depth and 0.2 μm filtration.

The polyclonal containing composition is digested using a low endotoxin papain and cysteine EDTA in a seventh step 113. Digesting can be stopped using iodoacetamide in an eighth step 115. The composition can be subjected to further depth filtration in a nineth step 117, and further filtered via ultrafiltration with phosphate buffered saline (PBL) in a tenth step 119 before in an eleventh step 121 additional depth and 0.2 μm filtration. The polyclonal containing composition can then be purified via ion exchange purification in a twelfth step 123, and further 0.2 μm ion exchange filtration. Once various filtrations and purification steps have been performed, the polyclonal containing composition can be concentrated and subjected to diafiltration in a thirteenth step 127, before a final 0.2 μm filtration in fourteenth step 129.

Preferably, the purity SEC of isolated antigen-binding fragments after the purifying is preferably >85%. Preferably, the purity RP of isolated antigen-binding fragments after the purifying is >85%. Preferably, the isolated antigen-binding fragments have a purity of >90% fab monomer. The isolated antigen-binding fragments have a molecular weight of about 40 kDa to 50 kDa.

Consistent with the above description, FABs from ovine were prepared and tested for efficacy.

To test efficacy, LD₅₀ ricin murine models were established at two different contract research organizations using two different ricin batches. Neutralization studies of ricin preincubated with varying doses of the obtained FAB were administered by the intraperitoneal (IP) route. In the first study, eight groups of Swiss Webster mice were exposed to either vehicle alone, the obtained FAB alone, ricin alone, or five different doses of the obtained FAB preincubated with ricin via the IP route. Mice were observed for weight change and mortality over a 4-day period. In the second study, two experiments of six groups of BALB/c mice were treated with vehicle alone, or a total of 7 different doses of the obtained FAB across two different experiments preincubated with ricin via the IP route. Body weight, clinical observations, and survival was monitored over a 14-day study period.

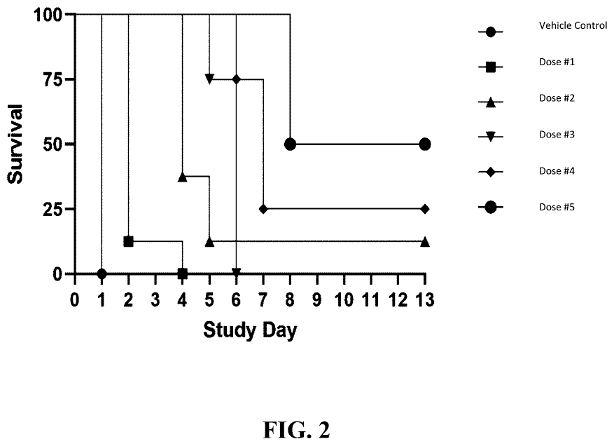

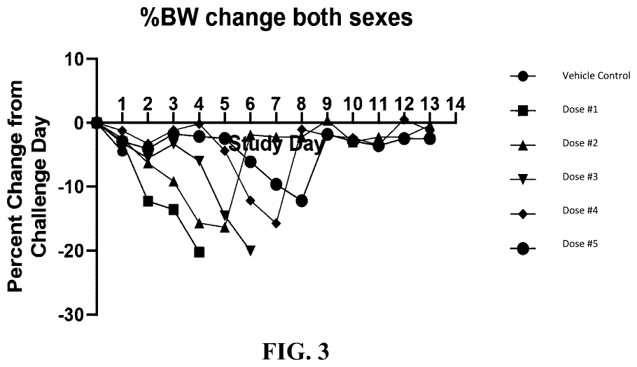

Animals challenged with the obtained FAB alone did not display any adverse clinical signs, minimal weight loss and overall weight gain by 96 hours. None of the mice in the first study exposed to all but the lowest dose of the obtained FAB preincubated for 30 minutes with ricin succumbed to the challenge. Some animals displayed weight loss and clinical signs, but these were less severe than in the ricin alone animals and mostly reversed by end of study. In the second study, the obtained FAB preincubated with ricin prior to IP administration appeared efficacious in a dose-dependent manner as evidenced by improved survival rates (FIG. 2), decreased incidence and severity of toxic signs and presentation of body weight loss following ricin challenge (FIG. 3).

Compared to ricin only controls, the obtained FAB incubated with ricin prior to IP administration appeared efficacious in a dose-dependent manner. Overall, the obtained FAB appeared well tolerated and proved efficacious at neutralizing ricin toxicity in two different IP mouse ricin models. This study shows the potential of the ovine platform as a scalable and viable solution to the lack of medical countermeasures to ricin toxicity.

As used herein, the term “an effective amount” or “therapeutic amount” refers to an amount of active ingredient which can illicit a desired response from a patient. In particular, such amounts are effective in curing or significantly reducing ricin intoxication when the active ingredient is used alone, or in combination with other active ingredients. As would be understood by a person of ordinary skill in the art, an effective amount can vary based on the amount of ricin involved in the intoxication.

As used herein, the term “active ingredient,” and the like, refer to products which provide a therapeutic effect on a patient.

Therapeutic amounts of Fabs according to the disclosure can be at least 10 mg; at least 100 mg; at least 250 mg; or at least 500 mg. In specific embodiments the effective amount can be from about 5 mg to 50,000 mg; about 5 mg to 20,500 mg; about 10 mg to about 1,000 mg; or about 550 mg. In some embodiments, the effective amount is less than 10,000 mg; less than 5,000 mg; or less than 1,000 mg.

In certain aspects of the disclosure, the Fabs can be characterized by their therapeutic effect. Fabs according to the disclosure can have an IC50 (ng/ml) of about 100 to 2,000; about 200 to 1,500; about 300 to 1,000; about 500 to about 1,000.

In some embodiments, the Fabs can neutralize ricin at a rate of about 20-100 ng of Fab to 1 ng of ricin or about 30-70 ng of Fab to 1 ng of ricin. The Fabs of the disclosure can neutralize ricin at a rate of less than 100 ng of Fab to 1 ng of ricin, less than 90 ng of Fab to 1 ng of ricin; less than 80 ng of Fab to 1 ng of ricin; or less than 70 ng of Fab to 1 ng of ricin.

Embodiments of the disclosure therefore include a pharmaceutical composition having as an active ingredient isolated antigen-binding fragments. The fragments can be obtained from ovine serum, and are capable of inhibiting/neutralizing ricin. In certain embodiments, the active ingredient can neutralize ricin at a rate of less than 100 ng to 1 ng of ricin.

To treat, or prevent from a subject at risk of ricin poisoning, the pharmaceutical composition is configured as an intravenous infusion. The pharmaceutical composition can include water, at least one electrolyte, and a buffer, and more specifically, for example, acetate and mannitol buffer. As used herein, the term “prevent” refers to prophylactic administration. Prophylactic administration includes continuous administration of the pharmaceutical composition. The continuous administration will depend on the total amount of expected exposure. Prevention can include infusion at 24 hr intervals. In some embodiments, the pharmaceutical composition is lyophilized. Lyophilized compositions can be diluted with saline or other carrier for administration.

In certain embodiments, the pharmaceutical composition contains about 40 mg to 50 grams of the active ingredient.

The following references are incorporated by reference:
Whitfield, S.J.C.; Griffiths, G.D.; Jenner, D.C.; Gwyther, R.J.; Stahl, F.M.; Cork, L.J.; Holley, J.L.; Green, A.C.; Clark, G.C. Production, Characterisation and Testing of an Ovine Antitoxin against Ricin; Efficacy, Potency and Mechanisms of Action. Toxins 2017, 9, 329.

Falach R, Sapoznikov A, Evgy Y, Aftalion M, Makovitzki A, Agami A, Mimran A, Lerer E, Ben David A, Zichel R, Katalan S, Rosner A, Sabo T, Kronman C, Gal Y. Post-Exposure Anti-Ricin Treatment Protects Swine Against Lethal Systemic and Pulmonary Exposures. Toxins (Basel). 2020 May 28;12(6):354.

Prigent J, Panigai L, Lamourette P, Sauvaire D, Devilliers K, Plaisance M, Volland H, Créminon C, Simon S. Neutralising antibodies against ricin toxin. PLOS One. 2011;6 (5):e20166. doi: 10.1371/journal.pone.0020166. Epub 2011 May 25.

1. A method of producing isolated antigen-binding fragments capable of neutralizing ricin at a rate of less than 100 ng to 1 ng of ricin, comprising:
immunizing a sheep with Ricinus communis-derived protein;
collecting blood from the sheep after the immunizing;
preparing a serum from the blood obtained in the collecting; and
purifying the serum to obtain isolated antigen-binding fragments.

2. The method of claim 1, wherein the Ricinus communis-derived protein includes ricin isoforms D, E, or the agglutinin RCA120.

3. The method of claim 1, wherein purifying further comprises digesting with papain.

4. The method of claim 1, wherein the immunizing includes a secondary immunization, and wherein the secondary immunization is about 28 days or about one-month after the first immunization.

5. The method of claim 1, wherein the purity SEC of isolated antigen-binding fragments after the purifying is >85%.

6. The method of claim 1, wherein the purity RP of isolated antigen-binding fragments after the purifying is >85%.

7. A pharmaceutical composition, comprising: as an active ingredient isolated antigen-binding fragments obtained from ovine serum and capable of inhibiting/neutralizing ricin, wherein the active ingredient can neutralize ricin at a rate of less than 100 ng to 1 ng of ricin.

8. The composition of claim 7, wherein the pharmaceutical composition is configured as an intravenous infusion.

9. The composition of claim 7, wherein the pharmaceutical composition is lyophilized.

10. The composition of claim 7, wherein the pharmaceutical composition contains about 40 mg to 50 grams of the active ingredient.

11. The composition of claim 7, wherein the composition further comprises acetate and mannitol buffer.

12. The composition of claim 7, wherein the isolated antigen-binding fragments have a molecular weight of about 40 kDa to 50 kDa.

13. The composition of claim 7, wherein the isolated antigen-binding fragments have a purity of >90% fab monomer.

14. A method of treating ricin poisoning, comprising: administering a pharmaceutically effective amount of the composition of claim 7 to a patient with ricin exposure.

15. The method of claim 14, wherein the composition of claim 1 is diluted in saline prior to the administering.

16. The method of claim 14, wherein the administering is by intravenous infusion.
